(19) United States
(12) Patent Application Publication
(10) Pub. No.: US 2025/0166749 A1
(43) Pub. Date: May 22, 2025

(54) WORKFLOW AUTOMATION WITH WEARABLE DEVICE

(71) Applicant: Bio-Rad Laboratories, Inc., Hercules, CA (US)

(72) Inventors: Jasmina DESAI, Irvine, CA (US); Shakila POTHINI, Hercules, CA (US)

(21) Appl. No.: 18/952,032

(22) Filed: Nov. 19, 2024

Related U.S. Application Data
(60) Provisional application No. 63/602,005, filed on Nov. 22, 2023.

(51) Int. Cl. G16H 10/40; G16H 40/67

(52) U.S. Cl. CPC G16H 10/40; G16H 40/67

(57) ABSTRACT

Systems and methods for automating hands-free workflow of laboratory procedures. The system includes a wearable device with a display and a workflow controller in communication with the wearable device. The workflow controller can be configured to extract a workflow for a specified laboratory procedure from a workflow repository. The workflow can include a plurality of instructions arranged in a specified sequence. One or more instructions of the plurality of instructions can be sequential delivered to the wearable device for an operator to follow. The one or more instructions can include a visual indicator configured to be displayed within the display of the wearable device. The wearable device can be configured to receive voice commands and the workflow controller can be configured to navigate the workflow based on the voice commands. The wearable device can be used to scan codes and display additional information during workflow implementation.

### CROSS-REFERENCE TO RELATED PATENT APPLICATIONS

The present application claims benefit of priority to U.S. Provisional Patent Application No. 63/602,005, filed on Nov. 22, 2023, which is incorporated by reference in its entirety for all purposes.

### BACKGROUND

Various medical procedures such as diagnostics, testing, etc. can be performed by operators such as clinicians or scientists in a laboratory setting. A procedure is typically printed on physical sheets as workflows. These printed sheets defining the workflow are used by clinicians or scientists to perform specified experiments or tests. For each step in the workflow, they have to refer back to the physical sheet that has a pre-defined workflow printed. This process involves a lot of manual work and takes a lot of time to go back and refer to the physical sheets after each step in the workflow.

### BRIEF SUMMARY

Workflows of laboratory procedures such as testing, biological sample collection, diagnostics, etc. involves several steps to be conducted in a particular sequence to successfully complete the procedure. Typically, the workflow steps are printed on a piece of paper or displayed on a computer screen. An operator has to go back and forth between the procedure to refer to the steps and instructions. Executing the workflow in this manner can be time consuming and error prone. Any missteps or error during performing an instruction of a workflow step may require reperforming the procedure or retracing several steps. This can be time consuming, may lead to waste of resources or scrapping of the entire test, or may generate inaccurate test results.

The present disclosure provides a wearable device configured to automate workflows guiding an operator through each step of the workflow. The wearable device facilitates hand-free access to the workflow steps and instructions so that the operator can focus on performing a task at hand efficiently and accurately. For example, the operator can perform tasks more accurately and efficiently because s/he does not have to stop, pick up (or scroll through on a computer) and read the paper instructions. The workflow steps and instructions can be conveyed via the wearable device in a specified sequence. As an example, the wearable device can be head wearable device or a head mounted display. For example, the head mounted device (e.g., eyeglasses) can include a small display coupled to a frame such that the display extends in front of one or both eyes of an operator (e.g., a scientist, clinician, technician, etc.). The display can be located within the field of vision of the operator such that s/he can look at the display directly without moving the head by directly gazing at the display. The display can be transparent or appropriately located in front of the eyes so the display does not obstruct the operator's vision while performing the tasks of the workflow. For example, a compact visual indication of workflow steps can be projected on an eyeglass display in the specified sequence. In some embodiments, at any given time, the operator can scan a QR code for retrieving information on a product by placing a field of view of the wearable device against the QR code of the product.

The wearable device-based workflow automation can advantageously save time during an installation process of a software (e.g., Unity Next Connect) used during a procedure (e.g., biological sample testing) so that the operator no longer have to refer back to the installation instructions. Instead, the operator can simply look through the wearable device itself. The example use cases herein illustrate different ways in which the wearable device herein can save a lot of manual work and time taken by scientists, lab users, install teams or other users, and automate the workflows for ease of use and better productivity. In many embodiments, navigating through the workflow can be voice enabled so that the operator (e.g., scientists) do not have to touch the wearable device while wearing gloves and performing the experiments/tests to avoid sample contamination. Navigation commands like left, right, up, down, next, cancel etc. can be integrated as voice or gesture-based commands with the wearable device to navigate through the workflow steps.

One aspect of the present disclosure relates to a system for automating hands-free workflow of laboratory procedures, according to various embodiments. The system includes a wearable device and a workflow controller. The wearable device can include a display for displaying instructions. The workflow controller in communication with the wearable device. The workflow controller can be configured to receive a selection of a laboratory procedure, extract a workflow for the selected laboratory procedure from a workflow repository. The workflow can include a plurality of instructions arranged in a specified sequence. One or more instructions of the plurality of instructions can be sequential delivered to the wearable device for an operator to follow. The one or more instructions can include a visual indicator configured to be displayed within the display of the wearable device.

In some embodiments, the visual indicator can include: a location indicator related to a location of a sample or a tool in a laboratory, an action indicator related to the laboratory procedure, and/or, data related to the sample or the tool. In some embodiments, these instructions can comprise voice instructions such as, for example, “next”, “previous”, “cancel”, or the like. In some embodiments, the one or more instructions can include an audio instruction related to a task of the laboratory procedure. The audio instruction being delivered simultaneously with the visual indicator.

In some embodiments, the wearable device is a head mounted device with the display positioned in a field of view of the operator. In some embodiments, the wearable device can further include a speaker configured to deliver an audio, and the workflow controller can be configured to generate audio instructions related to a task of the laboratory procedure, and deliver the audio instructions via the speaker of the wearable device.

In some embodiments, the wearable device can be further configured to receive a voice command from the operator to navigate between instructions of the plurality of instructions, and transmit the voice command to the workflow controller. The workflow controller can be configured to receive the voice command from the wearable device, and navigate within the workflow based on the voice command. In some embodiments, the voice command can include navigation commands to navigate through the plurality of instructions of the workflow in a hand-free manner. The navigation commands may include left, right, up, down, next, cancel, or a combination thereof.

In some embodiments, the wearable device can further include a camera for scanning a code from a sample container, and transmit the scanned code to the workflow controller. The workflow controller can be configured to retrieve, based on the scanned code, information related to the sample container, and transmit the information to the display of the wearable device. The information related to the sample container comprises: a batch number, an expiration date, a patient identifier, or the like.

In some embodiments, the wearable device can be further configured to capture, via the camera, an image of an object in the laboratory, and transmit the image to the workflow controller. The workflow controller can be configured to retrieve information related to the object, and transmit the information to the display of the wearable device.

In some embodiments, the laboratory procedure comprises a noninvasive prenatal testing (NIPT). For example, the plurality of instructions related to the NIPT can include: collect blood sample; separate plasma; extract cfDNA; prepare ddPCR assay; transfer the cfDNA to the ddPCR assay; generate a droplet; and analyze the droplet.

Another aspect of the present disclosure relates a method for automating hands-free workflow of laboratory procedures. The method can include receiving, via a computer, a selection of a laboratory procedure; extracting, from a workflow repository, a workflow for the selected laboratory procedure, where the workflow can include a plurality of instructions arranged in a specified sequence; sequential delivering one or more instructions of the plurality of instructions to a wearable device for an operator to follow, where the one or more instructions can include a visual indicator configured to be displayed within the display of the wearable device.

In some embodiments, the laboratory procedure comprises a noninvasive prenatal testing. For example, sequential delivering the one or more instructions can include: collecting blood sample; separating plasma; extracting cfDNA; preparing ddPCR assay; transferring the cfDNA to the ddPCR assay; generating a droplet; and analyzing the droplet.

In some embodiments, sequential delivering the one or more instructions can include delivering an audio instruction related to a particular workflow step, wherein the audio instruction is delivered simultaneously with the visual indicator. The visual indicator can include: a location indicator related to a location of a sample or a tool in a laboratory, an action indicator related to the laboratory procedure, and/or, data related to the sample or the tool.

In some embodiments, the method can further include receiving, via the wearable device, a voice command from the operator to navigate between instructions of the plurality of instructions, and transmit the voice command to the workflow controller, and navigating to a particular workflow step in the workflow based on the voice command.

In some embodiments, the method can further include capturing, via a camera of the wearable device, a code from a sample container; transmitting, via a wireless network, the captured code to the workflow controller; retrieving, via the workflow controller using the scanned code or the image, information related to the sample container or the object from database; and transmitting, via the wireless network, the information to the display of the wearable device.

The forgoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying figures are for illustration purposes only and can or cannot represent actual or preferred values or dimensions. Where applicable, some or all features cannot be illustrated to assist in the description of underlying features. In the drawings:

FIG. 1 is a schematic diagram of a system configured for conveying workflow steps of a laboratory procedure, in accordance with some embodiments of the present disclosure.

FIG. 2 illustrates an example wearable device of the system of FIG. 1 worn by an operator.

FIG. 3 is a container with scannable code identifiable by the wearable device of FIG. 2.

FIG. 4 is an example display system of the wearable device of FIG. 2.

FIG. 5 is a flow chart of an example noninvasive prenatal testing (NIPT) workflow executable via the system of FIG. 1.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Laboratory procedures can be laborious and involve several steps to be read off a printed sheet. An operator has to repeatedly pick up the sheet and read instructions before performing a task. There are no existing displays, wearable devices or other devices that are configured to efficiently deliver the workflows to the operator. In the following description, systems for improving laboratory workflow implementations are provided. The systems herein can automate a printed workflow by conveying workflow instructions in such a way that an operator can efficiently perform the laboratory procedure. For example, an operator can receive instructions automatically on a wearable device so that the operator does not have to pick and drop a printed sheet to read instructions, pick up a sample to look up an identifier, feed information into a computer to extract information about the sample or other objects. Thus, the operator can advantageously perform laboratory procedures in a more efficient manner, and avoid cross-contamination between samples, among other benefits.

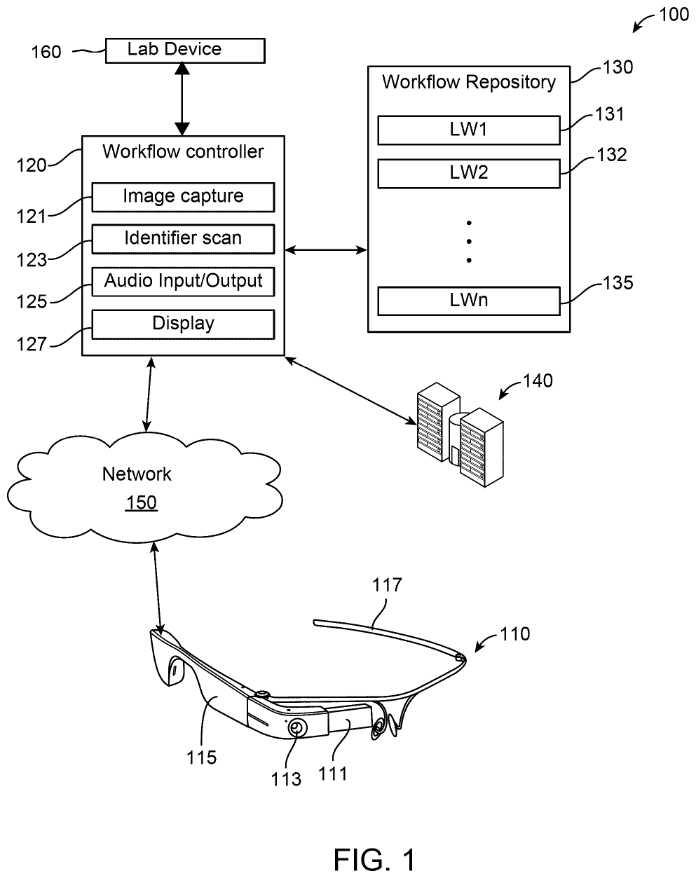

FIG. 1 is a schematic diagram of a system 100 configured for conveying workflow steps of a laboratory procedure, in accordance with some embodiments of the present disclosure. The system 100 can include a wearable device 110 configured to display workflow steps. The system 100 can include a workflow controller 120 configured to communicate via a wireless network 150 with the wearable device 110. Furthermore, the system 100 may include a workflow repository 130, a database 140, and/or a lab device 160. The lab device 160 can comprise a device controllable by a user to perform an operation, test, assay, analysis, or the like. In some embodiments, the lab device 160 can include a device for performing automated Polymerase Chain Reaction (PCR) and/or for performing automated digital PCR. This can include, for example, the device for performing automated digital PCR such as disclosed in International Application No. PCT/US2023/022567, filed on May 17, 2023, and entitled “SYSTEM AND METHOD FOR AUTOMATED DIGITAL POLYMERASE CHAIN REACTION”, the entirety of which is hereby incorporated by reference herein.

In some embodiment, the lab device 160 can communicate with the other components of the system 100. This can include, for example, communicating with the workflow coordinator 120 and/or the workflow repository 130. In some embodiments in which the workflow coordinator 120 is contained within the lab device 160, the lab device 160 can track the progression through the workflow, can track one or several past, present, and/or next steps in the workflow, and/or can control presentation of the present and/or next steps of the workflow to the user via the wearable device 110.

In some embodiments, the lab device 160 can detect completion of a step, can identify a next step, and can provide information relating to the next step to the user. In some embodiments, the providing of the next step to the user can include directing the wearable device 110 to provide instructions for completing the next step to the user. This can include, in some embodiments, triggering of a timer tracking time for completion of the next step, which timer can be visually or audibly presented to the user via the wearable device 110.

The workflow controller 120 can extract information from the workflow repository 130 and/or the database 140 based on the laboratory procedure. The workflow controller 120 may be a computer configured to communicate with the database 140 and the workflow repository 130 via a wired or a wireless connection. The workflow repository 130 and the database 140 may be updated as the laboratory setting change, workflow steps change or new workflows are developed, etc.

In many embodiments, the workflow repository 130 comprising laboratory workflows such as LW1 131, LW2 132, and LWn 135 associated with different laboratory procedures. For example, the laboratory workflows LW1-LWn can be related to noninvasive prenatal testing (NIPT), blood collection, protein separation, or other diagnostic or testing procedures. Additional example laboratory procedures are discussed in U.S. Pat. No. 11,578,322, which is incorporated herein by reference in its entirety. In some embodiments, the database 140 can be configured to store information related to patient data (e.g., patient ID), biological sample (e.g., blood), laboratory apparatus (e.g., centrifuge, speed), or other data specified in a workflow of a laboratory procedure.

In the present disclosure, each of the workflows LW1-LWn are configured to be include visual indicators and/or audio instructions to be delivered via the wearable device 110. Further, the system 100 is configured such that user inputs (e.g., QR code, voice command, etc.) can be received via the wearable device 110 to efficiently navigate the workflow instructions.

An exemplary workflow as part of digital droplet PCR (ddPCR) analysis is shown in Table 1, depicted in FIG. 6, which indicates processing steps that can be performed as part of preparation to perform a ddPCR assay. The system 100 can be used in connection with the workflow, and can assist a user in completing this workflow. The user can initiate the workflow via, for example, an interaction with the lab device 160, an interaction with the wearable device 110, and/or an interaction with another component of the system 100. Based on this interaction, information relating to the workflow can be retrieved, for example, by the workflow controller 120 from the workflow repository 130. In some embodiments, this information relating to the workflow can be retrieved form the lab device 160.

The workflow can include, for example, information identifying materials for use in each step of the workflow. The workflow can further include, for example, information identifying a time for completion of each step in the workflow and/or the time for completion of the workflow. The workflow can further include information indicating actions for completion of each of the steps in the workflow. As seen in Table 1, the workflow can include a turn-around-time (TAT), materials, and one or several processing steps. Although table 1 shows one set of exemplary steps, different processing steps could be performed in different assays and/or as parts of different assays.

Processing steps can be presented to the user in order that the processing steps are to be performed. The information relating to the processing steps can be provided via visual and/or audio to the user. In some embodiments, some or all of these processing steps can be further presented with information relating to a time for completion of the associated processing step. In some embodiments, this time can be in the form of a turn-around-time (TAT) for completion of the task.

In some embodiments, this time can be tracked by a timer which can be provided to the user via the wearable device 110. In some embodiments, the timer can be generated by the workflow coordinator 120, which can be, in some embodiments, a part of the lab device 160. In other words, in some embodiments, the workflow coordinator 120 can track the start of a step and the time to completion of the step, and the workflow coordinator 120 can control operation of the wearable device 110 such that the timer is provided to the user. In some embodiments, the timer can be generated by the wearable device 110, and specifically by one or more processors of the wearable device 110.

The provision of the timer by the wearable device and the ability of the wearable device 110 to provide and receive instructions in a contactless manner provides significant benefits. These include, for example, the decreasing the risk of contamination during performing of the workflow, simplification of the workflow by consolidating user interaction with the wearable device 110, increasing accuracy of timing steps, and facilitating timely completion of steps of the workflow.

In some embodiments, upon receipt of an indication of initiation of a processing step, the timer can be triggered. This indication of initiation of the processing step can be provided by the lab device 160, by the workflow coordinator 120, and/or by the user. In some embodiments, for example, the user can provide a verbal command detectable by the wearable device 160 that indicates start of the workflow.

The timer, which can comprise a countdown timer, can provide information to the user indicating an amount of time to completion of the processing step. In some embodiments, an upon the time for completion of the processing step expiring, the wearable device 110 can provide an audible and/or visual indicator of the expiration of the time for completion of the processing step. In some embodiments, this indicator can change as more time passes since the expiration of time for completion of the processing step. In some embodiments, for example, the indicator of the expiration of the time for completion of the processing step can include an audible alarm. As time passes since the expiration of time for completion of the processing step, the volume of the audible alarm can increase and/or a sound of the audible alarm, such as a frequency of the audible alarm can change to indicate the passing time.

In some embodiments, and as part of initiating the workflow, the user can provide information relating to attributes of the process being performed. This can include, for example, information identifying the number of processes being performed, more specifically, this information can identify the quantify of sample, and/or the number of assays being performed. For example, as part of initiation a workflow in which an assay is performed on a number of samples, such as, for example, eight samples, the user may provide information to the system 100 indicating that eight samples are being analyzed. In some embodiments, the system 100 can automatically update the duration of time indicated by the timer based on the information provided by the user. In some embodiments, the system 100 can further update information relating to equipment needed for performing the process to be performed. This can include, for example, providing information relating to the type and/or volume or reagents, enzymes, lysing agents, surfactants, and/or the like for performing the indicated process. In some embodiments, these adjustments based on information received from the user can be performed by the system 100, and in some embodiments, can be performed by the wearable device 110, the workflow controller 120, and/or the lab device 160.

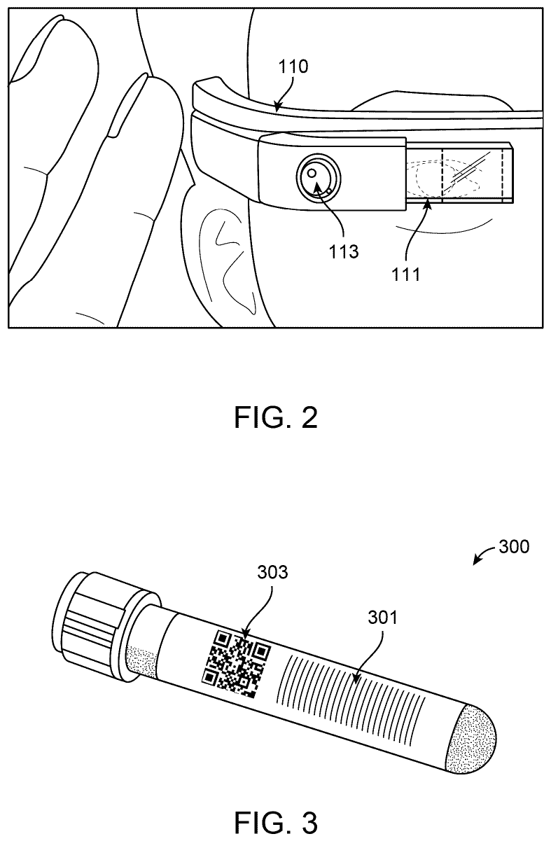

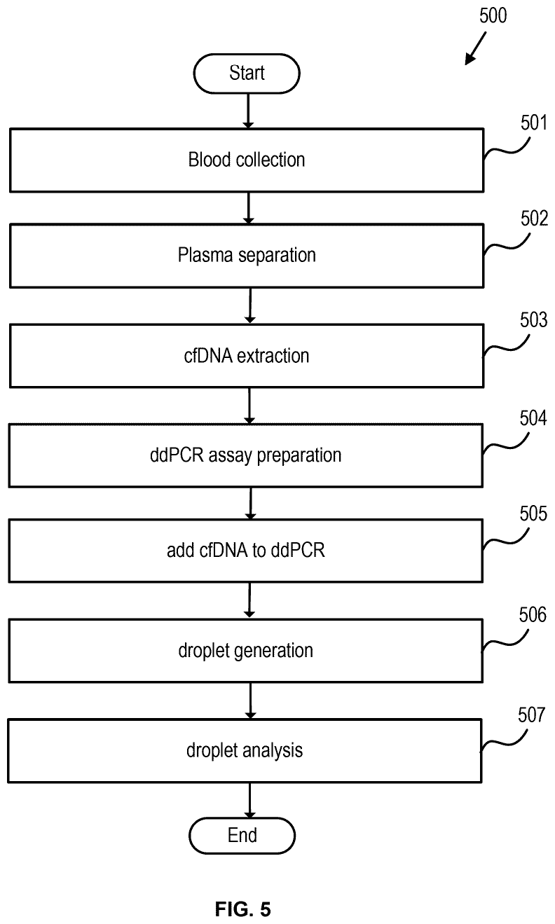

In the illustrated embodiments, the wearable device 110 can include a display 111, a camera 113, and a frame 115, 117. The display 111 can be coupled to the frame 115 such that a plane of the display, on which the laboratory workflow instructions can be projected, is directly within a field of vision of an operator (see FIG. 2). The camera 113 can be integrated with the frame 115. As an example, the display 111 can present information through visual, auditory, and/or other tactile cues (e.g., a display, a screen, a lamp, a light-emitting diode (LED), a graphical user interface (GUI), and the like). The frame 115 of the wearable device 110 may additionally house a computing system (e.g., a display system 400 in FIG. 4). The frame 115 may also include other electrical components and/or software components. The display 111 may be a see-through display, which is made of glass and/or another transparent or translucent material, such that the wearer can see their environment through the display 111. The present disclosure is not limited to a single display wearable device 110 and other wearable devices (e.g., eyeglasses) may be used. Additional examples of the wearable devices are described in U.S. Pat. Nos. 9,418,617; 9,568,734; 6,023,372; 9,128,285; and 10,936,872 all of which are incorporated herein by reference in their entirety. These wearable devices can be configured to, according to the present disclosure, automate a workflow so that an operator can perform a procedure efficiently in a substantially hand-free manner (e.g., without having to pick and drop a printed instruction sheet, pick up a sample, etc.).

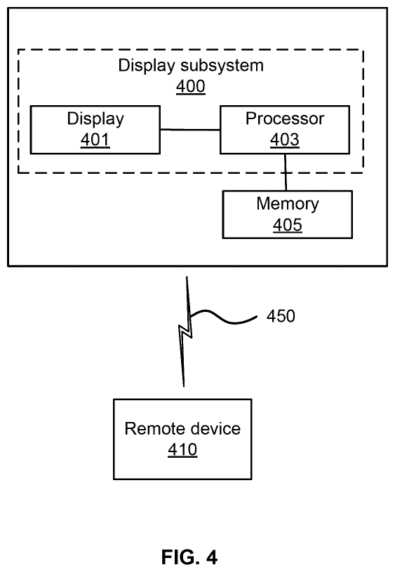

In some embodiments, the workflow controller 120 can include components configured to process data/instructions and facilitate interaction with the wearable device 110 via the network 150. For example, the workflow controller 120 can include, but is not limited to, an image capture module 121, an identifier scan module 123, an audio input/output module 125, and a display module 127. The image capture module 121 can communicate with the camera 113 of the wearable device 110 to receive an image, process the image, receive information about the objects in the image from the database 140. The processing of the image can involve determining if a workflow step (e.g., in the workflow LW1) in progress needs additional information about an object in the image. In another example, the operator may capture an image of an object via the camera 113 and request (e.g., via voice command) additional information about the object. Similarly, the identifier scan module 123 can receive a code (e.g., QR code 303, the bar code 301 in FIG. 3) via the camera 113. Further, the identifier scan module 123 can extract data related to the code. The additional information about an object or the code may be further configured via the display module 127 to be displayed on the display 113.

The display module 123 can be configured to generate visual indicators related to a particular step. The visual indicators can prompt the operator to proceed to a next workflow step, or go back to a previous step. The display module 123 may receive size information of the display 111 to appropriately configure the visual indicators to be fully displayed or multiple visual indicators to be displayed in a sequence. For example, the visual indicator may be a step identifier (e.g., a word, number, alphanumeric character), a left arrow indicating a previous step, a right arrow indicating to proceed to next step, a completion indicator, or other indicators to guide the operator through a laboratory workflow (e.g., LW1).

FIG. 2 illustrates the wearable device 110 of the system 100 head-mounted on an operator. The display 111 extends in front of the operator's eye to display workflow instructions (e.g., visual indications of a current step or a next step) without having to move the head to read the instructions projected on the display 111. In some embodiments, a short visual indication that can fit within the display 111 may be displayed to indicate a workflow step and details of the workflow step may be conveyed by audio. In some embodiments, a workflow step may involve scanning a code (e.g., QR code, barcode, etc.) to identify a biological sample. For example, FIG. 3. illustrates a container 300 with scannable codes 301, 303 identifiable via the camera 113 of the wearable device 110.

Based on the scanned code (e.g., 301, 303), additional information may be retrieved from a database 140 (see FIG. 1). The additional information may be displayed on the display 111 of the wearable device 110 or delivered as audio for the operator to complete the workflow step. Accordingly, the operator can complete various workflow steps more efficiently without getting interrupted to look or read instructions from a paper sheet, picking/placing an object, or accessing another computer to get additional information.

FIG. 4 is a block diagram of an example display subsystem 400 for a wearable device, according to various embodiments. For example, the display subsystem 400 may be embedded in the wearable device 110, according to an illustrated embodiment. The display subsystem 400 can communicate with a remote device 410 (e.g., the workflow controller 120 in FIG. 1) via a wireless network (e.g., 150 in FIG. 1). The display subsystem 400 can includes a display 401 (e.g., the display 111 of the wearable device 110) and a processor 403 configured to communicate with the workflow controller. The display 401 may be coupled with a frame of a wearable device or integrally formed on an eyeglass. In some embodiments, the display 401 may be removable, pivotable or fixed with respect to a frame of the wearable device. The display 401 may be, for example, an optical see-through display, an optical see-around display, or a video see-through display. In some embodiments, the processor 403 may receive information (e.g., workflow steps associated with a laboratory workflow, data, or other information) from the remote device 410 (e.g., the workflow controller 120). In some embodiments, the processor 403 may configure the instructions for display on the display 401. The processor 403 may be any type of processor, such as a micro-processor or a digital signal processor, for example.

The wearable device may further include data storage, such as memory 405 coupled to the processor 403. The memory 405 may store software that can be accessed and executed by the processor 403, for example.

A communication link 450 between the wearable device (e.g., 110) and the remote device (e.g., the workflow controller 120) may be a wireless connection such as the wireless network 150. For example, the communication link 450 may also be a wireless connection using, e.g., Bluetooth® radio technology, communication protocols described in IEEE 802.11 (including any IEEE 802.11 revisions), Cellular technology (such as GSM, CDMA, UMTS, EV-DO, WiMAX, or LTE), or Zigbee® technology, among other possibilities.

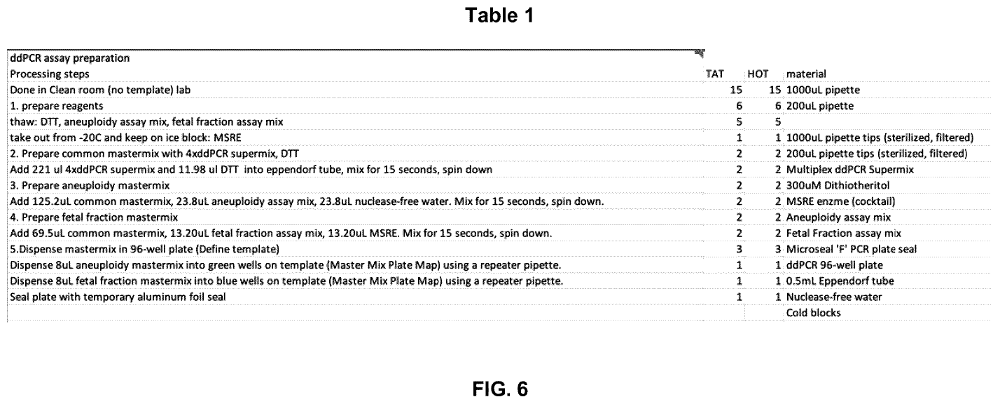

FIG. 5 is a flow chart of an example noninvasive prenatal testing (NIPT) workflow 500 executable via the system 100. For example, the workflow 500 can include steps 501-507. The steps can include visual and/or audio indicators to navigate through the NIPT workflow 500.

Step 501 may involve collecting blood sample. The blood sample may be stored in a container or collected from a patient. In some embodiments, for example, instructions for completing step 501 can be provided to the user via the wearable device 110. These instructions can include, for example, instructions for the type of container for holding the collected blood, instructions for collecting blood, instructions for the volume of blood to be connected, or the like.

Step 502 may involve separating plasma. In some embodiments, for example, instructions for completing step 502 can be provided to the user via the wearable device 110. These instructions can include, for example, instructions separating the plasma, including, for example, instructions for use of a centrifuge for separating the blood.

Step 503 may involve extracting cfDNA. In some embodiments, for example, instructions for completing step 503 can be provided to the user via the wearable device 110. These instructions can include, for example, for performing the DNA extraction, and specifically for performing the cfDNA extraction. These instructions can, for example, direct the user to mix one or several reagents, lysing agents, surfactants, or the like with all or portions of the sample.

Step 504 may involve preparing ddPCR assay. In some embodiments, for example, instructions for completing step 504 can be provided to the user via the wearable device 110. These instructions can include, for example, steps shown in Table 1.

Step 505 may involve adding cfDNA to ddPCR. Step 506 may involve generating droplet. Step 507 may involve analyzing droplet. In some embodiments, for example, instructions for completing step 505, 506, and/or step 507 can be provided to the user via the wearable device 110. In some embodiments, this can include providing instructions for operating a ddPCR system such as described in International Application No. PCT/US2023/022567, filed on May 17, 2023, and entitled “SYSTEM AND METHOD FOR AUTOMATED DIGITAL POLYMERASE CHAIN REACTION”, the entirety of which is hereby incorporated by reference herein. In some embodiments, instructions can be provided to the user via the wearable device 110, which instructions can correspond to instructions provided by a user interface of the ddPCR system.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

What is claimed is:

1. A system for automating hands-free workflow of laboratory procedures, the system comprising:
a wearable device comprising a display for displaying instructions; and
a workflow controller in communication with the wearable device, the workflow controller configured to:
receive a selection of a laboratory procedure;
extract, from a workflow repository, a workflow for the selected laboratory procedure, the workflow comprising a plurality of instructions arranged in a specified sequence; and
sequentially deliver one or more instructions of the plurality of instructions to the wearable device for an operator to follow, wherein the one or more instructions comprises a visual indicator configured to be displayed within the display of the wearable device.

2. The system of claim 1, wherein the wearable device is a head mounted device with the display positioned in a field of view of the operator.

3. The system of claim 1, wherein the one or more instructions comprises an audio instruction related to a task of the laboratory procedure, the audio instruction being delivered simultaneously with the visual indicator.

4. The system of claim 1, wherein the visual indicator comprises: a location indicator related to a location of a sample or a tool in a laboratory, an action indicator related to the laboratory procedure, and/or, data related to the sample or the tool.

5. The system of claim 1, wherein:
the wearable device further comprises a speaker configured to deliver an audio, and
the workflow controller is configured to generate audio instructions related to a task of the laboratory procedure, and deliver the audio instructions via the speaker of the wearable device.

6. The system of claim 1, wherein:
the wearable device is further configured to receive a voice command from the operator to navigate between instructions of the plurality of instructions, and transmit the voice command to the workflow controller, and
the workflow controller is configured to receive the voice command from the wearable device, and navigate within the workflow based on the voice command.

7. The system of claim 6, wherein the voice command comprises navigation commands to navigate through the plurality of instructions of the workflow in a hand-free manner.

8. The system of claim 7, wherein the navigation commands comprises left, right, up, down, next, and/or cancel.

9. The system of claim 1, wherein:
the wearable device further comprises a camera for scanning a code from a sample container, and transmit the scanned code to the workflow controller, and
the workflow controller is configured to retrieve, based on the scanned code, information related to the sample container, and transmit the information to the display of the wearable device.

10. The system of claim 9, wherein:
the information related to the sample container comprises: a batch number, an expiration date, a patient identifier.

11. The system of claim 9, wherein:
the wearable device is further configured to capture, via the camera, an image of an object in the laboratory, and transmit the image to the workflow controller, and
the workflow controller is configured to retrieve information related to the object, and transmit the information to the display of the wearable device.

12. The system of claim 1, wherein the laboratory procedure comprises a noninvasive prenatal testing (NIPT).

13. The system of claim 12, wherein the plurality of instructions related to the NIPT comprises:
collect blood sample;
separate plasma;
extract cfDNA;
prepare ddPCR assay;
transfer the cfDNA to the ddPCR assay;
generate a droplet; and
analyze the droplet.

14. The system of claim 1, further comprising a lab device configured to perform an assay.

15. The system of claim 14, wherein the lab device comprises the workflow controller.

16. The system of claim 15, wherein the workflow controller is configured to deliver instructions to the wearable device to cause the wearable device to provide a timer to a user, wherein the timer indicates an amount of time for successful completion of a current step in the workflow.

17. A method for automating hands-free workflow of laboratory procedures, the method comprising:
receiving, via a computer, a selection of a laboratory procedure;
extracting, from a workflow repository, a workflow for the selected laboratory procedure, the workflow comprising a plurality of instructions arranged in a specified sequence; and
sequential delivering one or more instructions of the plurality of instructions to a wearable device for an operator to follow, wherein the one or more instructions comprises a visual indicator configured to be displayed within the display of the wearable device.

18. The method of claim 17, wherein the laboratory procedure comprises a noninvasive prenatal testing.

19. The method of claim 18, wherein sequential delivering the one or more instructions comprises:
collecting blood sample;
separating plasma;
extracting cfDNA;
preparing ddPCR assay;
transferring the cfDNA to the ddPCR assay;
generating a droplet; and
analyzing the droplet.

20. The method of claim 17, wherein sequential delivering the one or more instructions comprises delivering an audio instruction related to a particular workflow step, wherein the audio instruction is delivered simultaneously with the visual indicator.

21. The method of claim 17, wherein the visual indicator comprises: a location indicator related to a location of a sample or a tool in a laboratory, an action indicator related to the laboratory procedure, and/or, data related to the sample or the tool.

22. The method of claim 17, further comprising:
receiving, via the wearable device, a voice command from the operator to navigate between instructions of the plurality of instructions, and transmit the voice command to the workflow controller; and
navigating to a particular workflow step in the workflow based on the voice command.

23. The method of claim 17, further comprising
capturing, via a camera of the wearable device, a code from a sample container;
transmitting, via a wireless network, the captured code to the workflow controller;
retrieving, via the workflow controller using the captured code or an image, information related to the sample container from database; and
transmitting, via the wireless network, the information to the display of the wearable device.
